(19) United States
(12) Patent Application Publication
(10) Pub. No.: US 2024/0074694 A1
(43) Pub. Date: Mar. 7, 2024

(54) SKIN STATE ESTIMATION METHOD, DEVICE, PROGRAM, SYSTEM, TRAINED MODEL GENERATION METHOD, AND TRAINED MODEL

(71) Applicant: SHISEIDO COMPANY, LTD., Tokyo (JP)

(72) Inventors: Noriko HASEGAWA, Tokyo (JP); Yuusuke HARA, Tokyo (JP); Takuma HOSHINO, Tokyo (JP)

(21) Appl. No.: 18/262,620

(22) PCT Filed: Feb. 15, 2022
(86) PCT No.: PCT/JP2022/005909

§ 371 Date: Jul. 24, 2023

(30) Foreign Application Priority Data
Feb. 15, 2021 (JP) 2021-021916

(51) Int. Cl. A61B 5/00

(52) U.S. Cl. CPC A61B 5/441

(57) ABSTRACT

A skin state is readily obtained. A method according to one embodiment of the present invention includes identifying a nasal feature of a user; and estimating a skin state of the user based on the nasal feature of the user.

### TECHNICAL FIELD

The present invention relates to a skin state estimation method, a device, a program, a system, a trained model generation method, and a trained model.

### BACKGROUND ART

Conventionally, for appropriate care or the like of skin, a technique to predict a skin state is known. For example, PTL 1 uses ultrasonic images to predict future formation of wrinkles around the eyes and the mouth and levels of the wrinkles.

### CITATION LIST

### Patent Literature

[PTL 1] Japanese Laid-Open Patent Publication No. 2011-200284

### SUMMARY OF INVENTION

### Technical Problem

PTL 1, however, needs an ultrasonic diagnostic device, and thus it is not easy to simply predict a skin state that is likely to occur in the future.

In view of the above, an object of the present invention is to readily know about a skin state.

### Solution to Problem

A method according to one embodiment of the present invention includes identifying a nasal feature of a user, and estimating a skin state of the user based on the nasal feature of the user.

### Advantageous Effects of Invention

In the present invention, it is possible to readily estimate a skin state from a nasal feature.

### BRIEF DESCRIPTION OF DRAWINGS

FIG. 1 illustrates an overall configuration according to one embodiment of the present invention.

FIG. 2 is a functional block diagram of a skin state estimation device according to one embodiment of the present invention.

FIG. 3 is a flowchart of a skin state estimation process according to one embodiment of the present invention.

FIG. 4 is an explanatory view illustrating a nasal feature according to one embodiment of the present invention.

FIG. 5 is an explanatory view of extraction of a nose region according to one embodiment of the present invention.

FIG. 6 is an explanatory view of calculation of a nasal feature value according to one embodiment of the present invention.

FIG. 7 is one example of a nasal feature of a face type according to one embodiment of the present invention.

FIG. 8 is one example of a face estimated from a nasal feature according to one embodiment of the present invention.

FIG. 9 illustrates a hardware configuration of a skin state estimation device according to one embodiment of the present invention.

### DESCRIPTION OF EMBODIMENTS

Hereinafter, embodiments of the present invention will be described with reference to the drawings. Note that, in the present specification and drawings, components having substantially the same function and configuration are given the same symbols, and duplicate descriptions thereof are omitted.

### <Explanation of Terms>

The “skin state” refers to a wrinkle, a spot, sagging, dark circles, a nasolabial fold, dullness, elasticity, moisture, sebum, melanin, blood circulation, a blood vessel, blood, pore, a skin color, or any combination thereof. For example, the “skin state” refers to the presence or absence of or the extent of an element that constitutes such a skin state as a wrinkle, a spot, sagging, dark circles, a nasolabial fold, dullness, elasticity, moisture, sebum, melanin, blood circulation, a blood vessel, blood, pore, and a skin color. Also, the “skin state” refers to a skin state in a part of the face, the whole face, or a plurality of sites in the face. Note that, the “skin state” may be a future skin state of a user or a current skin state of a user. In the present invention, the skin state is estimated from a nasal feature based on a correlation between the nasal feature and the skin state.

### <Overall Configuration>

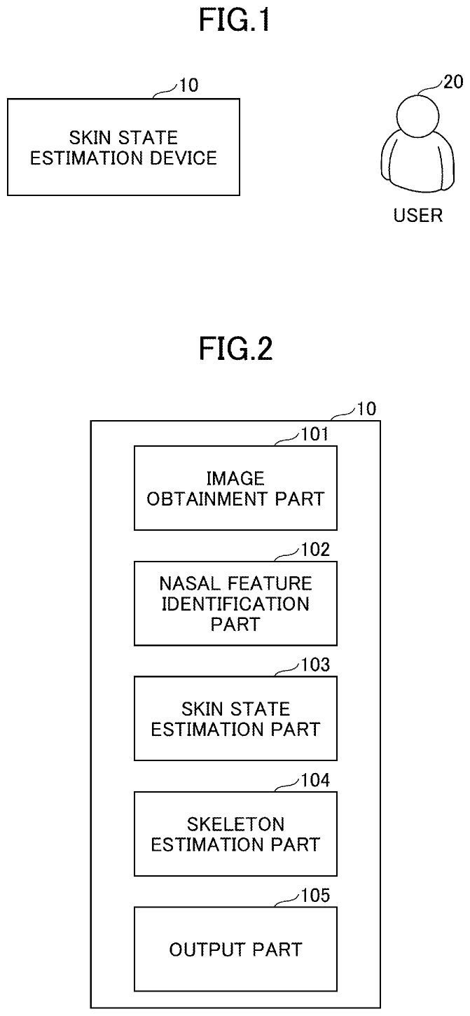

FIG. 1 illustrates an overall configuration according to one embodiment of the present invention. A skin state estimation device estimates a skin state of a user 20 from a nasal feature of the user 20. The skin state estimation device 10 is, for example, a smartphone having a camera function. In the following, referring to FIG. 2, the skin state estimation device 10 will be described in detail.

Note that, in the present specification, a case in which the skin state estimation device 10 is a single device (e.g., a smartphone having a camera function) will be described, but the skin state estimation device 10 may be composed of a plurality of devices (e.g., a smartphone having no camera function and a digital camera). Also, the camera function may be a function of photographing skin three-dimensionally or a function of photographing skin two-dimensionally. Also, a device other than the skin state estimation device 10 (e.g., a server) may execute a part of the process that is executed by the skin state estimation device 10 as described in the present specification.

### <Functional Blocks of Skin State Estimation Device 10>

FIG. 2 is a functional block diagram of the skin state estimation device 10 according to one embodiment of the present invention. The skin state estimation device 10 can include an image obtainment part 101, a nasal feature identification part 102, a skin state estimation part 103, a skeleton estimation part 104, and an output part 105. Also, by executing a program, the skin state estimation device 10 can function as the image obtainment part 101, the nasal feature identification part 102, the skin state estimation part 103, the skeleton estimation part 104, and the output part 105. In the following, each of the parts will be described.

The image obtainment part 101 obtains the image including the nose of the user 20. Note that, the image including the nose may be an image obtained by photographing the nose and parts other than the nose (e.g., an image obtained by photographing the whole face) or may be an image obtained by photographing only the nose (e.g., an image obtained by photographing a nose region of the user 20 so as to be within a predetermined region displayed on a display device of the skin state estimation device 10). Note that, when the nasal feature is identified from something other than the image, the image obtainment part 101 is not needed.

The nasal feature identification part 102 identifies the nasal feature of the user 20. For example, the nasal feature identification part 102 identifies the nasal feature of the user 20 from image information of the image including the nose of the user 20 obtained by the image obtainment part 101 (the image information is, for example, a pixel value of the image).

The skin state estimation part 103 estimates the skin state of the user 20 based on the nasal feature of the user 20 identified by the nasal feature identification part 102. For example, the skin state estimation part 103 classifies the skin state of the user 20 based on the nasal feature of the user 20 identified by the nasal feature identification part 102.

Note that, the skin state estimation part 103 can also estimate the skin state of the user 20 based on the shape of a facial skeleton of the user 20 estimated by the skeleton estimation part 104 (e.g., the skin state attributed to the shape of a facial skeleton).

The skeleton estimation part 104 estimates the shape regarding the facial skeleton of the user 20 based on the nasal feature of the user 20 identified by the nasal feature identification part 102. For example, the skeleton estimation part 104 classifies the shape regarding the facial skeleton of the user 20 based on the nasal feature of the user 20 identified by the nasal feature identification part 102.

The output part 105 outputs (e.g., displays) information of the skin state of the user estimated by the skin state estimation part 103.

### <Skin State>

Here, the skin state will be described. For example, the skin state is a wrinkle, a spot, sagging, dark circles, a nasolabial fold, dullness, elasticity, moisture, sebum, melanin, blood circulation, a blood vessel, blood, texture, pore, a skin color, or any combination thereof. More specifically, the skin state is, for example, a wrinkle at the corner of the eye, a wrinkle under the eye, a wrinkle on the forehead, a wrinkle in the eye socket, sagging of the eye bag, dark circles under the eyes, a nasolabial fold (a nasolabial sulcus, a line around the mouth), a depth of a nasolabial sulcus, sagging of a marionette line, sagging of the jaw, HbSO2 Index (hemoglobin oxygen saturation index), Hb Index (hemoglobin level), HbO2 (oxyhemoglobin level), skin tone, skin brightness, transepidermal water loss (TEWL), the number of skin bumps, viscoelasticity of skin, a blood oxygen level, a vascular density, the number of micro-blood vessels, the number of branched blood vessels, a distance between the blood vessels and the epidermis, a thickness of the epidermis, HDL cholesterol, sebum, a moisture content, a melanin index (indicator of melanin), pore, transparency, color unevenness (brownish color, reddish color), pH, or the like. The skin state estimation part 103 estimates the skin state from the nasal feature based on the correlation between the nasal feature and the skin state.

### <Correspondence Relationship Between the Nasal Feature and the Skin State>

Here, the correspondence relationship between the nasal feature and the skin state will be described. The skin state estimation part 103 estimates the skin state based on the correspondence relationship between the nasal feature and the skin state that is previously stored in, for example, the skin state estimation device 10. Note that, the skin state estimation part 103 may estimate the skin state based on not only the nasal feature but also the nasal feature and a part of a face feature.

The correspondence relationship may be a predetermined database or a trained model generated through machine learning. In the database, the nasal feature (which may be the nasal feature and a part of the face feature) and the skin state are associated with each other based on, for example, results of experiments conducted on test subjects. Meanwhile, the trained model is a prediction model that outputs information of the skin state in response to an input of information of the nasal feature (which may be the nasal feature and a part of the face feature).

### <<Generation of Trained Model>>

In one embodiment of the present invention, a computer such as the skin state estimation device 10 can generate a trained model. Specifically, the computer such as the skin state estimation device 10 obtains training data including input data that are nasal features (which may be nasal features and parts of face features) and output data that are skin states. Through machine learning using the training data, it is possible to generate a trained model that outputs a skin state in response to an input of a nasal feature (which may be a nasal feature and a part of a face feature). In this way, through machine learning using the training data including input data that are nasal features (which may be nasal features and parts of the face features) and output data that are skin states, a trained model that outputs a skin state in response to an input of a nasal feature (which may be a nasal feature and a part of a face feature) is generated.

### <Correspondence Relationship Between the Shape Regarding the Facial Skeleton and the Skin State>

Here, the correspondence relationship between the shape regarding the facial skeleton and the skin state will be described. As described above, the skin state estimation part 103 can also estimate the skin state based on the correspondence relationship between the shape regarding the facial skeleton and the skin state that is previously stored in, for example, the skin state estimation device 10.

The correspondence relationship may be a predetermined database or a trained model generated through machine learning. In the database, the shape regarding the facial skeleton and the skin state are associated with each other based on, for example, results of experiments conducted on test subjects. Meanwhile, the trained model is a prediction model that outputs information of the skin state in response to an input of information of the shape regarding the facial skeleton.

### <<Generation of Trained Model>>

In one embodiment of the present invention, a computer such as the skin state estimation device 10 can generate a trained model. Specifically, the computer such as the skin state estimation device 10 obtains training data including input data that are shapes regarding skeletons of the faces and output data that are skin states. Through machine learning using the training data, it is possible to generate a trained model that outputs a skin state in response to an input of a shape regarding a facial skeleton. In this way, through machine learning using the training data including input data that are shapes regarding facial skeletons and output data that are skin states, a trained model that outputs a skin state in response to an input of a shape regarding a facial skeleton is generated.

### <Future Skin State and Current Skin State>

Note that, the skin state to be estimated may be a future skin state of the user 20 or a current skin state of the user 20. When the correspondence relationship between the nasal feature (or the shape regarding the facial skeleton estimated from the nasal feature) and the skin state is made based on data of people who have ages higher than the actual age of the user 20 (e.g., the ages of the test subjects for the experiments or the ages of people who provide training data for machine learning are higher than the actual age of the user 20), the future skin of the user 20 is estimated. Meanwhile, when the correspondence relationship between the nasal feature (or the shape regarding the facial skeleton estimated from the nasal feature) and the skin state is made based on data of people who have the same ages as the actual age of the user 20 (e.g., the ages of the test subjects for the experiments or the ages of people who provide training data for machine learning are the same as the actual age of the user 20), the current skin of the user 20 is estimated. Note that, the skin state may be estimated based on not only the nasal feature but also the nasal feature and a part of the face feature.

In the following, estimation examples will be described. Each of the estimation examples is based on the correspondence relationship between the nasal feature (or the shape regarding the facial skeleton estimated from the nasal feature) and the skin state.

### <<Estimation Example 1 of the Skin State>>

For example, the skin state estimation part 103 can estimate that when the nasal root and the nasal bridge are high, wrinkles are more likely to form at the corners of the eyes. Also, for example, the skin state estimation part 103 can estimate that when the cheeks have such shapes that high cheekbones are located at upper parts of the cheeks, there are wrinkles at the corners of the eyes or there is a possibility that wrinkles are likely to form in the future (determination of ON/OFF).

### <<Estimation Example 2 of the Skin State>>

For example, the skin state estimation part 103 can estimate that when the nasal wings are more rounded or when, for example, the eyes are large, wrinkles are more likely to form under the eyes.

The orbits have shape-related features, such as a horizontally long shape and a small shape. However, for example, the skin state estimation part 103 can estimate that when the orbits are large and have such shapes that the vertical and horizontal widths thereof are close to each other, there are many wrinkles under the eyes. Also, for example, the skin state estimation part 103 can estimate wrinkles under the eyes based on the face outline. Also, for example, the skin state estimation part 103 can estimate that when the distance between the eyes is longer, there are a smaller number of wrinkles under the eyes.

### <<Estimation Example 3 of the Skin State>>

For example, the skin state estimation part 103 can estimate sagging of the eye bags based on the roundness of the nasal wings and the height of the nasal bridge. Specifically, the skin state estimation part 103 can estimate that when the sum of the roundness of the nasal wings and the height of the nasal bridge is larger, the eye bags are sagging.

For example, the skin state estimation part 103 can estimate that when the face outline is oval and the face is long, the eye bags are more likely to sag.

### <<Estimation Example 4 of the Skin State>>

For example, the skin state estimation part 103 can estimate HbCO2 (reduced hemoglobin) based on how low the nasal bridge is and on the roundness of the nasal wings.

For example, the skin state estimation part 103 can estimate HbSO2 (oxygen saturation) based on the face outline.

### <<Estimation Example 5 of the Skin State>>

For example, the skin state estimation part 103 can estimate that when the nasal bridge is lower, the nasal wings are more rounded, or the distance between the eyes is larger, the moisture content is lower.

For example, the skin state estimation part 103 can estimate the moisture content of skin based on how high the cranial index is and on an aspect ratio of the face.

### <<Estimation Example 6 of the Skin State>>

For example, the skin state estimation part 103 can estimate sebum based on the roundness of the nasal wings.

For example, the skin state estimation part 103 can estimate sebum based on the face outline.

### <<Estimation Example 7 of the Skin State>>

For example, the skin state estimation part 103 can estimate that when the nasal wings are more rounded and the nasal bridge is higher, the melanin index is higher and the amount of melanin is larger, and that when the nasal bridge is lower and the distance between the eyes is shorter, the melanin index is lower.

For example, the skin state estimation part 103 can estimate that when both of the upper lip and the lower lip are thicker, the melanin index is higher and the amount of melanin is larger. Also, for example, the skin state estimation part 103 can estimate that when both of the upper lip and the lower lip are thinner, the melanin index is lower.

### <<Estimation Example 8 of the Skin State>>

For example, the skin state estimation part 103 can estimate that when the nasal wings are round, dark circles under the eyes are more likely to form.

### <<Estimation Example 9 of the Skin State>>

For example, the skin state estimation part 103 can estimate that when the nasal bridge is low and the distance between the eyes is relatively long or when the angle of the jaw is round, the face outline is more likely to sag.

### <<Estimation Example 10 of the Skin State>>

For example, the skin state estimation part 103 can estimate that when the nasal bridge is higher, the blood oxygen content is higher.

### <<Estimation Example 11 of the Skin State>>

For example, the skin state estimation part 103 can estimate the vascular density from the size of the nasal wings or the position at which the nasal root begins to change in height. The larger the nasal wings are, the higher the vascular density is.

### <<Estimation Example 12 of the Skin State>>

For example, the skin state estimation part 103 can estimate the thickness of the epidermis from the size of the nasal wings.

### <<Estimation Example 13 of the Skin State>>

For example, the skin state estimation part 103 can estimate the number of branched blood vessels from the position at which the nasal root begins to change in height.

### <<Comprehensive Estimation of the Skin State>>

In one embodiment of the present invention, the skin state estimation part 103 can comprehensively represent the skin state from the values estimated in, for example, the above Estimation examples 1 to 9, as a wrinkle, a spot, sagging, dark circles, a nasolabial fold, dullness, elasticity, moisture, sebum, melanin, blood circulation, a blood vessel, blood, pore, or a skin color. One example is given below.
Wrinkle: represented by one item or two or more items of a wrinkle at the corner of the eye, a wrinkle under the eye, a wrinkle on the forehead, and a wrinkle in the eye socket.
Spot: represented by one item or two or more items of color unevenness in brown, color unevenness in reddish color, and melanin.
Sagging: represented by one item or two or more items of an eye bag, the jaw, and a marionette line.
Dark circles: represented by one or two items of brown dark circles under the eyes and blue dark circles under the eyes.
Nasolabial fold: represented by one or two items of a nasolabial fold in a nasolabial sulcus and a nasolabial fold around the mouth.
Dullness: represented by one item or two or more items of transparency, melanin, color unevenness, a skin color, oxygen saturation, moisture, and the number of skin bumps.
Elasticity: represented by one item or two or more items of moisture, sebum, sagging, and viscoelasticity of skin.
Moisture: represented by one or two items of a moisture content, transepidermal water loss (TEWL), the number of skin bumps, and pH.
Texture: represented by one item or two or more items of the number of skin bumps and moisture.
Skin color: represented by one item or two or more items of skin tone, skin brightness, melanin, a blood oxygen level, and HbO2 (oxyhemoglobin level).
Sebum: represented by one or two items of an amount of sebum and pore.
   Note that, normal skin, dry skin, oily skin, and combination skin may be classified based on moisture and sebum.
Melanin: represented by one or two items of a melanin index, an amount of melanin, and color unevenness.
Blood circulation: represented by at least one or two items of HbSO2 Index (hemoglobin oxygen saturation index), Hb Index (hemoglobin level), HbO2 (oxyhemoglobin level), a blood oxygen level, and a skin color.
Blood vessel: represented by one item or two or more items of a vascular density, the number of micro-blood vessels, the number of branched blood vessels, a distance between the blood vessels and the epidermis, and a thickness of the epidermis.
Blood: HDL cholesterol.

In one embodiment of the present invention, the skin state estimation part 103 can represent skin features, such as skin strength and skin weakness, from the nasal feature. For example, the nasal feature that is type 1 is representative of the skin strength because the evaluation value of the wrinkle at the corner of the eye is lower than the average evaluation value. The nasal feature that is type 2 is representative of the skin weakness because the evaluation value of the wrinkle at the corner of the eye is higher than the average evaluation value. The skin strength and weakness can be represented from place to place on the face. In the case of Type 1, the skin strength includes a wrinkle or spot at the corner of the eye and a wrinkle or spot on the forehead, and the skin weakness includes dark circles, a nasolabial fold in a nasolabial sulcus, sagging around the mouth, and water retainability. The skin state estimation part 103 can estimate a comprehensive indicator of skin (in this case, skin of a sagging type) from these skin states. In the case of Type 2, the skin strength includes sagging of cheeks, water retainability, blood circulation, and a spot, and the skin weakness includes a wrinkle or spot at the corner of the eye and a wrinkle or spot on the forehead. The skin state estimation part 103 can estimate a comprehensive indicator of skin (in this case, skin of a wrinkle type) from these skin states.

### <Shape Regarding Facial Skeleton>

Here, the shape regarding the facial skeleton will be described. The “shape regarding the facial skeleton” refers to a shape of a facial skeleton itself, a face shape attributed to the skeleton, or both. Based on the correlation between the nasal feature and the shape regarding the facial skeleton, the skeleton estimation part 104 estimates the shape regarding the facial skeleton from the nasal feature.

For example, the shape regarding the facial skeleton is a feature of a bone shape, a positional relationship of a skeleton, an angle, or the like in the orbits, the cheekbones, the nose bone, the piriform aperture (the opening of the nose cavity opened toward the face), the cephalic index, the maxilla, the mandible, the lips, the corners of the mouth, the eyes, the epicanthal folds (skin folds existing in portions where the upper eyelids cover the inner corners of the eyes), the face outline, the positional relationships between the eyes and the eyebrows (e.g., the eyes and the eyebrows are far away or are near), or any combination thereof. In the following, one example of the shape regarding the facial skeleton will be given. Note that, exemplary specifics that can be estimated are described in parentheses.
Orbit (horizontally long, square, rounded)
Cheekbone, cheek (peak position, roundness)
Nose bone (width, shape)
Piriform aperture (shape)
Cephalic index (width/depth of the cranial bone=70, 75, 80, 85, 90)
Maxilla, upper jaw (positional relationship with the orbit, nasolabial angle)
Mandible, lower jaw (length of the depth, angle in the depth, forward angle, contour shape (defined jawline))
Forehead (roundness of the forehead, shape of the forehead)
Eyebrow (distance between the eye and the eyebrow, shape of the eyebrow, thick eyebrow)
Lip (both of the upper lip and the lower lip are thick, the lower lip is thick, both of the upper lip and the lower lip are thin, horizontally large or small)
Corners of the mouth (going up or down, standard)
Eye (area, angle, distance between the eyebrow and the eye, distance between the eyes)
Epicanthal fold (present, absent)
Face outline (Rectangle, Round, Oval, Heart, Square, Average, Natural, Long)

### <Correspondence Relationship Between the Nasal Feature and the Shape Regarding the Facial Skeleton>

Here, the correspondence relationship between the nasal feature and the shape regarding the facial skeleton will be described. Based on the correspondence relationship between the nasal feature and the shape regarding the facial skeleton that is previously stored in, for example, the skin state estimation device 10, the skeleton estimation part 104 estimates the shape regarding the facial skeleton. Note that, the skeleton estimation part 104 may estimate the shape regarding the facial skeleton based on not only the nasal feature but also the nasal feature and a part of the face feature.

The correspondence relationship may be a predetermined database or a trained model generated through machine learning. In the database, the nasal feature (which may be the nasal feature and a part of the face feature) and the shape regarding the facial skeleton are associated with each other based on, for example, results of experiments conducted on test subjects. Meanwhile, the trained model is a prediction model that outputs information of the shape regarding the facial skeleton in response to an input of information of the nasal feature (which may be the nasal feature and a part of the face feature). Note that, the correspondence relationship between the nasal feature and the shape regarding the facial skeleton may be made for each of the populations classified based on factors that can influence their skeletons (e.g., Caucasoid, Mongoloid, Negroid, and Australoid).

### <<Generation of Trained Model>>

In one embodiment of the present invention, a computer such as the skin state estimation device 10 can generate a trained model. Specifically, the computer such as the skin state estimation device 10 obtains training data including input data that are nasal features (which may be nasal features and parts of the face features) and output data that are shapes regarding facial skeletons. Through machine learning using the training data, it is possible to generate a trained model that outputs a shape regarding a facial skeleton in response to an input of a nasal feature (which may be a nasal feature and a part of the face feature). In this way, through machine learning using the training data including input data that are nasal features (which may be nasal features and parts of the face features) and output data that are shapes regarding facial skeletons, a trained model that outputs a shape regarding a facial skeleton in response to an input of a nasal feature (which may be a nasal feature and a part of the face feature) is generated.

In the following, estimation examples will be described. Each of the estimation examples is based on the correspondence relationship between the nasal feature and the shape regarding the facial skeleton.

### <<Estimation Example 1 of the Shape Regarding the Facial Skeleton>>

For example, the skeleton estimation part 104 can estimate the cranial index based on how high or low the nasal root is or the position at which the nasal root begins to change in height, and on how high or low the nasal bridge is. Specifically, the skeleton estimation part 104 estimates that when the nasal root, the nasal bridge, or both are higher, the cranial index is lower.

### <<Estimation Example 2 of the Shape Regarding the Facial Skeleton>>

For example, the skeleton estimation part 104 can estimate that the corners of the mouth are going up or down based on the width of the nasal bridge. Specifically, the skeleton estimation part 104 estimates that when the width of the nasal bridge is larger, the corners of the mouth go down.

### <<Estimation Example 3 of the Shape Regarding the Facial Skeleton>>

For example, the skeleton estimation part 104 can estimate how large and thick the lip is (1. both of the upper and lower lips are large and thick, 2. the lower lip is thick, 3. both of the upper and lower lips are thin and small) based on roundness of the nasal wings and sharpness of the nasal tip.

### <<Estimation Example 4 of the Shape Regarding the Facial Skeleton>>

For example, the skeleton estimation part 104 can estimate presence or absence of the epicanthal folds based on the nasal root. Specifically, the skeleton estimation part 104 estimates that when the nasal root is determined to be low, the epicanthal folds are present.

### <<Estimation Example 5 of the Shape Regarding the Facial Skeleton>>

For example, the skeleton estimation part 104 can classify the shape of the lower jaw (e.g., into three) based on how low or high the nasal bridge is, how high the nasal root is, and how round and large the nasal wings are.

### <<Estimation Example 6 of the Shape Regarding the Facial Skeleton>>

For example, the skeleton estimation part 104 can estimate the piriform aperture based on how high the nasal bridge is.

### <<Estimation Example 7 of the Shape Regarding the Facial Skeleton>>

For example, the skeleton estimation part 104 can estimate the distance between the eyes based on how low the nasal bridge is. Specifically, the skeleton estimation part 104 estimates that when the nasal bridge is lower, the distance between the eyes is longer.

### <<Estimation Example 8 of the Shape Regarding the Facial Skeleton>>

For example, the skeleton estimation part 104 can estimate roundness of the forehead based on how high the nasal root is and how high the nasal bridge is.

### <<Estimation Example 9 of the Shape Regarding the Facial Skeleton>>

For example, the skeleton estimation part 104 can estimate the distance between the eye and the eyebrow, and the shape of the eyebrow based on how high or low the nasal bridge is, how large the nasal wings are, and the position at which the nasal root begins to change in height.

### <Processing Method>

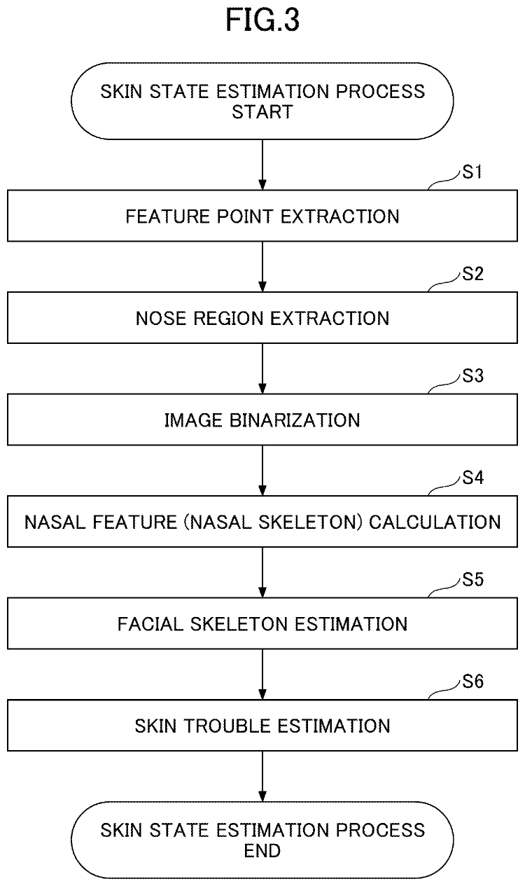

FIG. 3 is a flowchart of a skin state estimation process according to one embodiment of the present invention.

In step 1 (S1), the nasal feature identification part 102 extracts a feature point from an image including the nose (e.g., a feature point of the head of the eyebrow, the inner corner of the eye, or the tip of the nose).

In step 2 (S2), the nasal feature identification part 102 extracts a nose region based on the feature point that is extracted in S1.

Note that, when the image including the nose is an image obtained by photographing only the nose (e.g., an image obtained by photographing a nose region of the user 20 so as to be within a predetermined region displayed on a display device of the skin state estimation device 10), the image obtained by photographing only the nose is used as it is (i.e., S1 can be omitted).

In step 3 (S3), the nasal feature identification part 102 reduces the number of gradations of the image of the nose region that is extracted in S2 (e.g., binarizes the image). For example, the nasal feature identification part 102 reduces the number of gradations of the image of the nose region using brightness, luminance, Blue of RGB, Green of RGB, or any combination thereof. Note that, S3 can be omitted.

In step 4 (S4), the nasal feature identification part 102 identifies the nasal feature (nasal skeleton). Specifically, the nasal feature identification part 102 calculates a nasal feature value based on image information of the image of the nose region (e.g., a pixel value of the image). For example, the nasal feature identification part 102 calculates, as the nasal feature value, the average of the pixel values of the nose region, the number of pixels that is less or more than or equal to a predetermined value, the cumulative pixel value, the amount of change of the pixel value, or the like.

In step 5 (S5), the skeleton estimation part 104 estimates the shape regarding the facial skeleton. Note that, S5 can be omitted.

In step 6 (S6), the skin state estimation part 103 estimates the skin state (e.g., skin trouble in the future) based on the nasal feature identified in S4 (or the shape regarding the facial skeleton estimated in S5).

### <Feature of the Nose>

Here, the nasal feature will be described. For example, the nasal feature is the nasal root, the nasal bridge, the nasal tip, the nasal wings, or any combination thereof.

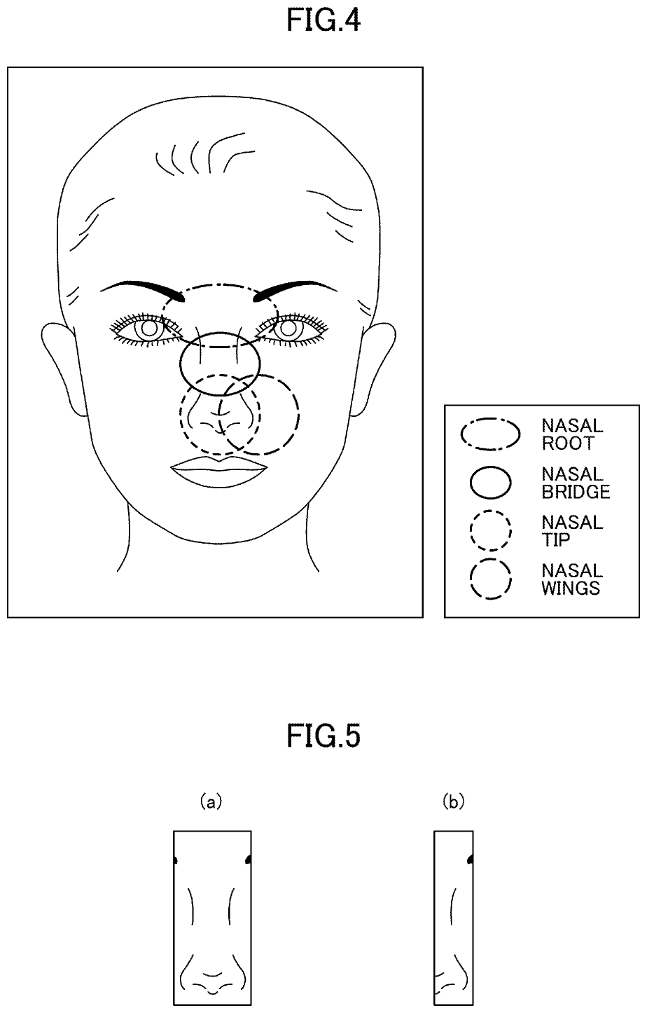

FIG. 4 is an explanatory view illustrating the nasal feature according to one embodiment of the present invention. FIG. 4 illustrates the positions of the nasal root, the nasal bridge, the nasal tip, and the nasal wings.

### <<Nasal Root>>

The nasal root is a region of the base of the nose. For example, the nasal feature is how high the nasal root is, how low the nasal root is, how wide the nasal root is, changing of the nasal root to become higher, the position at which the nasal root begins to change, or any combination thereof.

### <<Nasal Bridge>>

The nasal bridge is a region between the inter-eyebrow region and the nasal tip. For example, the nasal feature is how high the nasal bridge is, how low the nasal bridge is, how wide the nasal bridge is, or any combination thereof.

### <<Nasal Tip>>

The nasal tip is a tip portion of the nose (the tip of the nose). For example, the nasal feature is roundness or sharpness of the nasal tip, the direction of the nasal tip, or any combination thereof.

### <<Nasal Wings>>

The nasal wings are lateral round parts at both sides of the tip of the nose. For example, the nasal feature is roundness or sharpness of the nasal wings, how large the nasal wings are, or any combination thereof.

### <Extraction of the Nose Region>

FIG. 5 is an explanatory view of extraction of the nose region according to one embodiment of the present invention. The nasal feature identification part 102 extracts the nose region in the image including the nose. For example, the nose region may be the whole nose as illustrated in (a) of FIG. 5, or may be a part of the nose (e.g., the right or left half) as illustrated in (b) of FIG. 5.

### <Calculation of Nasal Feature Value>

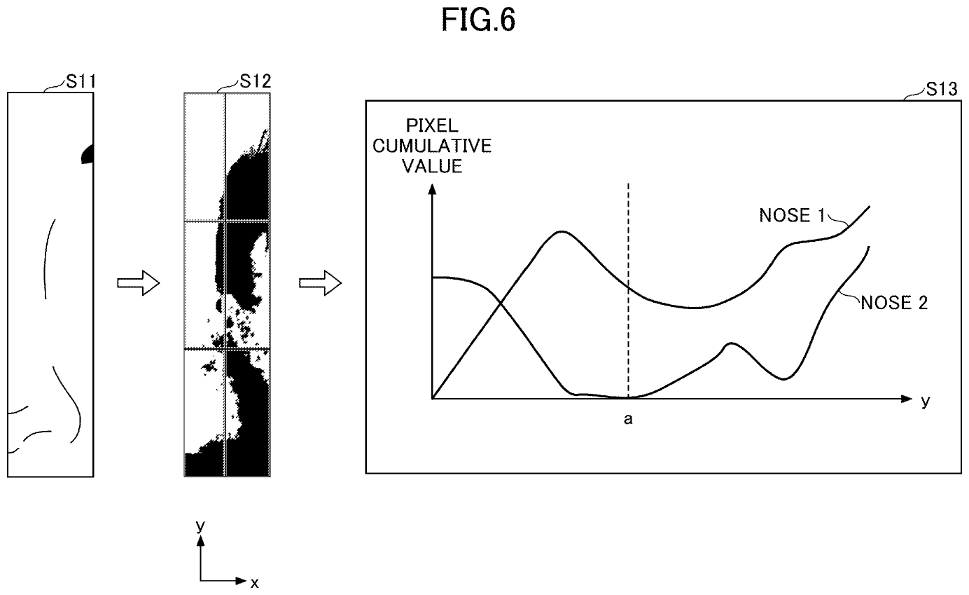

FIG. 6 is an explanatory view of calculation of the nasal feature value according to one embodiment of the present invention.

In step 11 (S11), the nose region in the image including the nose is extracted.

In step 12 (S12), the number of gradations of the image of the nose region extracted in S11 is reduced (for example, binarized). Note that, S12 can be omitted.

In step 13 (S13), the nasal feature value is calculated. Note that, in FIG. 6, the cumulative pixel value is represented with the higher brightness side and the lower brightness side of the image being 0 and 255, respectively. For example, the nasal feature identification part 102 performs normalization for each of a plurality of regions (e.g., the divided regions of S12). Next, for each of the regions, the nasal feature identification part 102 calculates, as the nasal feature value, the average of the pixel values, the number of pixels that is less or more than or equal to a predetermined value, the cumulative pixel value in the X direction, the Y direction, or both of the directions, the amount of change of the pixel value in the X direction, the Y direction, or both of the directions, or the like (e.g., using the data at the higher or lower brightness side of the image). In S13 of FIG. 6, the cumulative pixel value in the X direction is calculated at each of the positions in the Y direction.

In the following, how to calculate each of the feature values will be described.

For example, the nasal feature value root is a feature value of the upper region (closer to the eyes) in the divided regions of S12, the nasal feature value bridge is a feature value of the upper or middle region in the divided regions of S12, and the feature values of the nasal tip and the nasal wings are feature values of the lower region (closer to the mouth) in the divided regions of S12. These nasal feature values are normalized by the distance between the eyes.
Height of the nasal root: how high or low the nasal root is judged from the amount of change in the pixel value in the Y direction in the upper nose region. Note that, how high or low the nasal root may be calculated as a numerical value, or may be classified as being high or low. Regarding the position at which the nasal root begins to change in height, Nose 2 in S13 drastically changes in the value in the Y direction, indicating that the position at which the nasal root begins to change in height is at an upper part.
Width of the nasal root: the upper nose region is divided into two or more (e.g., two to four) in the X direction, and the width of the nasal root is judged from a pattern of the average of the pixel values in each region.
Height of the nasal bridge: how high or low the nasal bridge is judged from the average of the cumulative pixel values in the middle nose region. Note that, how high or low the nasal bridge may be calculated as a numerical value, or may be classified as being high or low.
Width of the nasal bridge: the middle nose region is divided into two or more (e.g., two to four) in the X direction, and the width of the nasal bridge is judged from a pattern of the average of the pixel values in each region.
Roundness or sharpness of the nasal tip: it is determined from other nasal features (the height of the nasal bridge, the roundness or sharpness of the nasal wings), and when the nasal bridge is lower and the nasal wings are more rounded, the nasal tip is more rounded.
Direction of the nasal tip: it is determined from the width from the lowermost point of the nose at a position corresponding to a predetermined percentage of the maximum value of the cumulative pixel value in the X direction in the middle nose region, and when the above width is larger, the nasal tip faces upward.
Roundness or sharpness of the nasal wings: it is judged from the amount of change in the value in the Y direction in the lower nose region.
Size of the nasal wings: it is judged from a percentage of the number of pixels equal to or less than a predetermined value in the middle region of the lower region. When the number of pixels is larger, the nasal wings are larger.

### <<Face Type>>

As described above, the “shape regarding the facial skeleton” refers to a “shape of a facial skeleton itself”, a “face shape attributed to the skeleton”, or both. The “shape regarding the facial skeleton” can include face types.

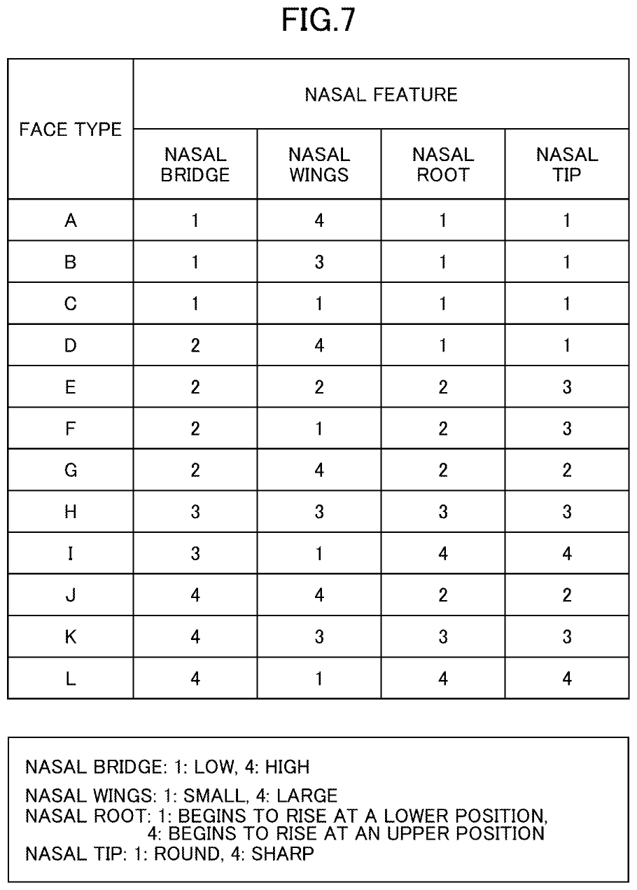

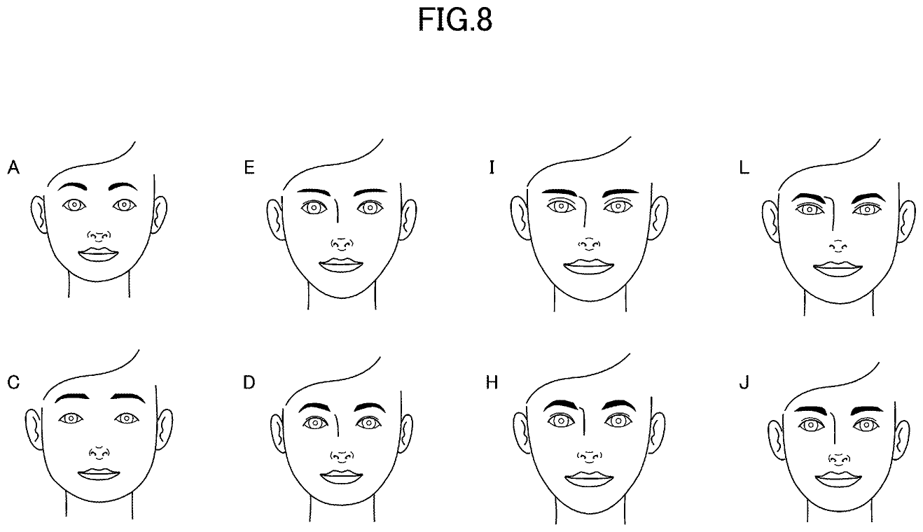

In one embodiment of the present invention, it is possible to estimate, based on the user's nasal feature, which face type of two or more face types the user's face is (specifically, the two or more face types are classified based on the “shape of a facial skeleton itself”, the “face shape attributed to the skeleton”, or both). In the following, the face types will be described with reference to FIG. 7 and FIG. 8.

FIG. 7 is one example of the nasal feature of each face type according to one embodiment of the present invention. FIG. 7 illustrates the nasal feature of each face type (each of Face types A to L). Note that, the face type may be estimated using all (four) of the nasal bridge, the nasal wings, the nasal root, and the nasal tip, or the face type may be estimated using one or some of them (e.g., the nasal bridge and the nasal wings (two of them), the nasal bridge and the nasal root (two of them), the nasal bridge alone, the nasal wings alone).

In this way, the face type is estimated from the nasal feature. For example, from the nasal feature of Face type A, the following are estimated; i.e., the roundness of the eyes: round, the tilt of the eyes: downward, the size of the eyes: small, the shape of the eyebrows: arch shape, the positions of the eyebrows and the eyes: far away, and the face outline: ROUND. Also, for example, from the nasal feature of Face type L, the following are estimated; i.e., the roundness of the eyes: sharp, the tilt of the eyes: considerably upward, the size of the eyes: large, the shape of the eyebrows: sharp, the positions of the eyebrows and the eyes: considerably near, and the face outline: RECTANGLE.

FIG. 8 is one example of the face estimated from the nasal feature according to one embodiment of the present invention. In one embodiment of the present invention, it is possible to estimate, based on the user's nasal feature, which face type of various face types as illustrated in FIG. 8 the user's face is.

In this way, it is possible to classify the face type from the nasal feature value that does not readily receive effects of lifestyle habits and a situation upon the photographing. For example, the face type classified based on the nasal feature can be utilized for suggesting a guidance for makeup and properties of skin (for example, it is possible to suggest a guidance for makeup and properties of skin based on what face features the chosen face type has and on what impressions the chosen face type gives).

### <Effects>

In this way, in the present invention, it is possible to readily estimate the skin state from the nasal feature. In one embodiment of the present invention, by estimating a future skin state from the nasal feature, it is possible to select cosmetics that can more effectively reduce skin trouble in the future, and determine a beauty treatment such as massaging.

### <Hardware Configuration>

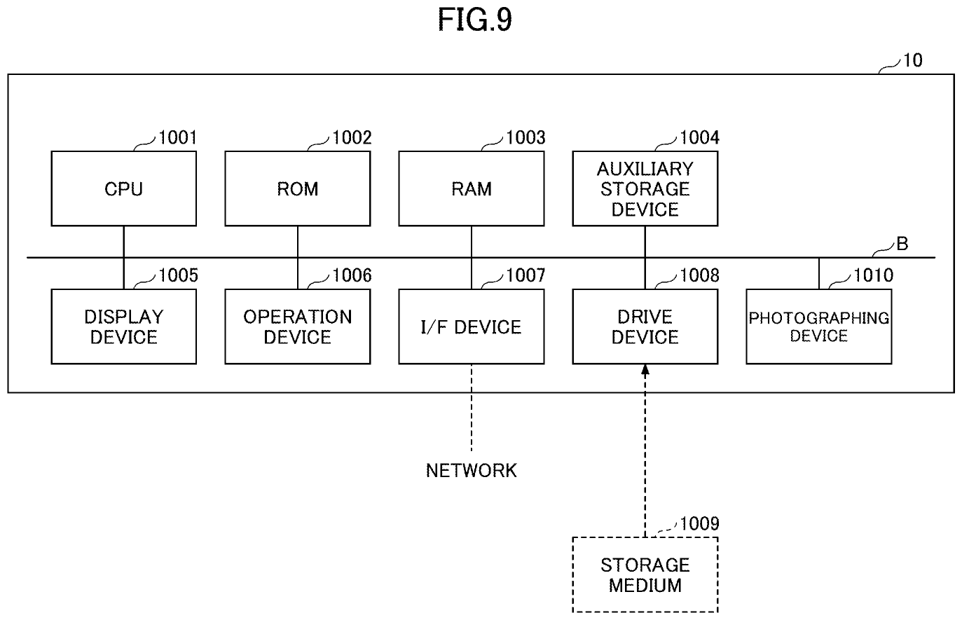

FIG. 9 illustrates a hardware configuration of the skin state estimation device 10 according to one embodiment of the present invention. The skin state estimation device 10 includes a CPU (Central Processing Unit) 1001, a ROM (Read Only Memory) 1002, and a RAM (Random Access Memory) 1003. The CPU 1001, the ROM 1002, the RAM 1003 forms what is called a computer.

Also, the skin state estimation device 10 can include an auxiliary storage device 1004, a display device 1005, an operation device 1006, an I/F (Interface) device 1007, and a drive device 1008.

Note that, the hardware components of the skin state estimation device 10 are connected to each other via a bus B.

The CPU 1001 is an arithmetic logic device that executes various programs installed in the auxiliary storage device 1004.

The ROM 1002 is a non-volatile memory. The ROM 1002 functions as a main storage device that stores, for example, various programs and data necessary for the CPU 1001 to execute the various programs installed in the auxiliary storage device 1004. Specifically, the ROM 1002 functions as a main storage device that stores, for example, boot programs such as BIOS (Basic Input/Output System) and EFI (Extensible Firmware Interface).

The RAM 1003 is a volatile memory such as DRAM (Dynamic Random Access Memory) or SRAM (Static Random Access Memory). The RAM 1003 functions as a main storage device that provides a working area developed when the various programs installed in the auxiliary storage device 1004 are executed by the CPU 1001.

The auxiliary storage device 1004 is an auxiliary storage device that stores various programs and information used when the various programs are executed.

The display device 1005 is a display device that displays, for example, an internal state of the skin state estimation device 10.

The operation device 1006 is an input device with which an operator of the skin state estimation device 10 inputs various instructions to the skin state estimation device 10.

The I/F device 1007 is a communication device that is connected to a network and is for communication to other devices.

The drive device 1008 is a device in which a storage medium 1009 is set. As used herein, the storage medium 1009 includes media that optically, electrically, or magnetically record information like a CD-ROM, a flexible disc, a magneto-optical disc, and the like. Also, the storage medium 1009 may include, for example, semiconductor memories that electrically record information like an EPROM (Erasable Programmable Read Only Memory), a flash memory, and the like.

Note that, the various programs installed in the auxiliary storage device 1004 are installed by, for example, setting the provided storage medium 1009 to the drive device 1008 and reading out various programs recorded in the storage medium 1009 by the drive device 1008. Alternatively, the various programs installed in the auxiliary storage device 1004 may be installed by downloading those programs from a network via the I/F device 1007.

The skin state estimation device 10 includes a photographing device 1010. The photographing device 1010 photographs the user 20.

While examples of the present invention have been described above in detail, the present invention is not limited to the above-described specific embodiments, and various modifications and changes are possible within the scope of the gist of the present invention described in the scope of claims.

The present international application claims priority to Japanese Patent Application No. 2021-021916, filed on Feb. 15, 2021. The contents of Japanese Patent Application No. 2021-021916 are incorporated in the present international application by reference in their entirety.

### DESCRIPTION OF THE REFERENCE NUMERAL

10 skin state estimation device
20 user
101 image obtainment part
102 nasal feature identification part
103 skin state estimation part
104 skeleton estimation part
105 output part
1001 CPU
1002 ROM
1003 RAM
1004 auxiliary storage device
1005 display device
1006 operating device
1007 I/F device
1008 drive device
1009 storage medium
1010 photographing device

1. A skin state estimation method, comprising:
identifying a nasal feature of a user; and
estimating a skin state of the user based on the nasal feature of the user.

2. The skin state estimation method according to claim 1, further comprising obtaining an image including a nose of the user,
wherein the nasal feature of the user is identified from image information of the image.

3. The skin state estimation method according to claim 1, wherein the skin state of the user is a future skin state of the user.

4. The skin state estimation method according to claim 1, wherein the skin state is a wrinkle, a spot, facial sagging, dark circles, a nasolabial fold, dullness of skin, elasticity, moisture, sebum, melanin, blood circulation, a blood vessel, blood properties, texture of skin, pore of skin, a skin color, or any combination thereof.

5. The skin state estimation method according to claim 4, further comprising estimating a comprehensive indicator of skin from the skin state.

6. The skin state estimation method according to claim 1, wherein the skin state is a skin state in a part of a face, a whole face, or a plurality of sites in a face.

7. The skin state estimation method according to claim 1, further comprising estimating a shape regarding a facial skeleton of the user based on the nasal feature of the user,
wherein the estimation of the skin state of the user is based on the shape regarding the facial skeleton of the user.

8. The skin state estimation method according to claim 7, wherein the skin state of the user is attributed to the shape regarding the facial skeleton of the user.

9. The skin state estimation method according to claim 1, wherein the nasal feature is a nasal root, a nasal bridge, a nasal tip, nasal wings, or any combination thereof.

10. The skin state estimation method according to claim 1, wherein the skin state of the user is estimated using a trained model that outputs the skin state in response to an input of the nasal feature.

11. A skin state estimation device, comprising:
an identification part configured to identify a nasal feature of a user; and
an estimation part configured to estimate a skin state of the user based on the nasal feature of the user.

12. A non-transitory computer-readable recording medium storing a program that causes a computer to execute a process comprising:
identifying a nasal feature of a user; and
estimating a skin state of the user based on the nasal feature of the user.

13.-15. (canceled)
